(19) United States
(12) Patent Application Publication
(10) Pub. No.: US 2011/0217257 A1
(43) Pub. Date: Sep. 8, 2011

(54) NOVEL 4-ALKYL CYCLOHEXANEPROPANAL COMPOUNDS AND THEIR USE IN PERFUME COMPOSITIONS

(76) Inventors: Robert P. Belko, Monroe, NJ (US); Anthony T. Levorse, JR., Westfield, NJ (US); Michael G. Monteleone, Hazlet, NJ (US)

(21) Appl. No.: 13/109,032

(22) Filed: May 17, 2011

Related U.S. Application Data
(63) Continuation-in-part of application No. 12/619,325, filed on Nov. 16, 2009.

(51) Int. Cl. C07C 47/11; A61K 8/33; C11D 3/50; A61L 9/01

(52) U.S. Cl. 424/76.2; 568/420; 512/22; 514/772; 510/106

(57) ABSTRACT

The present invention is directed to novel 4-alkyl cyclohexanepropanal compounds and a method of improving, enhancing or modifying a fragrance formulation through the addition of an olfactory acceptable amount of the novel compounds.

### STATUS OF RELATED APPLICATIONS

This application is a continuation-in-part of U.S. Ser. No. 12/619,325, filed on Nov. 16, 2009, the contents hereby incorporated by reference as if set forth in its entirety.

### FIELD OF THE INVENTION

The present invention relates to new chemical entities and the incorporation and use of the new chemical entities as fragrance materials.

### BACKGROUND OF THE INVENTION

There is an ongoing need in the fragrance industry to provide new chemicals to give perfumers and other persons the ability to create new fragrances for perfumes, colognes and personal care products. Those with skill in the art appreciate how small differences in chemical structures can result in unexpected and significant differences in odor, notes and characteristics of molecules. These variations allow perfumers and other persons to apply new compounds in creating new fragrances. For example, benzene compounds that differ slightly in substituents possess completely different odor profiles [Ishikawa, et al., International Journal of Quantum Chemistry 79: 101-108 (2000)]. In the case of tert-butyl cyclohexanes, the odor is said to be dependent on the compounds' conformation and therefore analogs adopting same conformation possess similar odor. Accordingly, many trans-compounds are shown to share pronounced urine-perspiration-type odor, while the corresponding cis-compounds are odorless or at the most possess weak and undefinable flowery or woody odor. However, some other trans- and cis-tert-butyl cyclohexanes are shown to possess opposite sensory activities [Ohloff, et al., Helvetica Chimica Acta 66, Fasc. 5: 1343-1354 (1983)]. Thus, it is hard for those with skill in the art to predict a given structure would be effective in sensory activities. Identifying desirable fragrance chemicals continues to pose difficult challenges.

### SUMMARY OF THE INVENTION

The present invention provides novel chemicals and their unexpected advantageous use in enhancing, improving or modifying the fragrance of perfumes, colognes, toilet waters, fabric care products, personal products and the like.

More specifically, the present invention is directed to novel 4-isobutyl cyclohexanepropanal compounds that exhibit unexpected strong fragrance effect, particularly a floral muguet like odor, and a method of improving, enhancing or modifying a fragrance formulation through the addition of an olfactory acceptable amount of 4-isobutyl cyclohexanepropanals represented by Formula I set forth below:

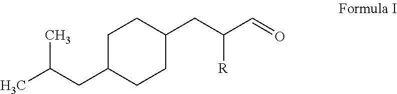

wherein R is selected from the group consisting of hydrogen and methyl.

These and other embodiments of the present invention will be apparent by reading the following specification.

### DETAILED DESCRIPTION OF THE INVENTION

It is known to those with the skill in the art that Formula I as defined above provides the following novel compounds:

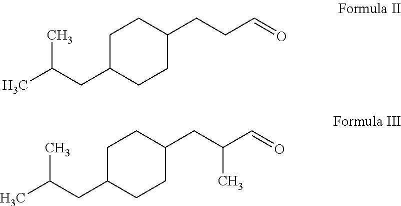

Those with the skill in the art will recognize that:

Formula II represents 4-isobutyl cyclohexanepropanal; and

Formula III represents α-methyl-4-isobutyl cyclohexanepropanal.

The compounds of the present invention can be prepared from 3-(4-isobutyl-phenyl)-propionaldehydes (commercially available). The reaction steps can be depicted by the scheme shown as follows:

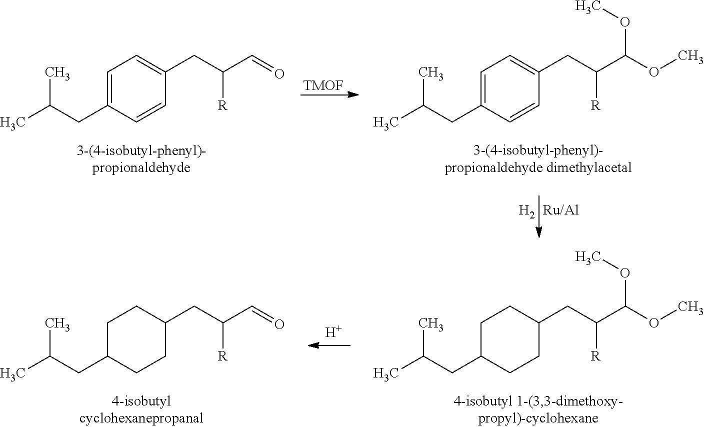

wherein R is defined as above;

TMOF represents trimethyl orthoformate; and

Ru/Al represents ruthenium on alumina catalyst.

Those with skill in the art will recognize that the 1,4-substituted aromatic rings contained in the compounds of the present invention give rise to a number of trans- and cis-isomers. It is intended herein that the compounds described herein include isomeric mixtures of such compounds, as well as those isomers that may be separated using techniques known to those having skill in the art. Suitable techniques include chromatography such as high performance liquid chromatography, referred to as HPLC, and particularly gel chromatography and solid phase microextraction, referred to as SPME.

The use of the compounds of the present invention is widely applicable in current perfumery products, including the preparation of perfumes and colognes, the perfuming of personal care products such as soaps, shower gels, and hair care products, fabric care products as well as air fresheners and cosmetic preparations. These compounds can also be used to perfume cleaning agents, such as, but not limited to detergents, dishwashing materials, scrubbing compositions, window cleaners and the like. In these preparations, the compounds of the present invention can be used alone or in combination with other perfuming compositions, solvents, adjuvants and the like. The nature and variety of the other ingredients that can also be employed are known to those with skill in the art.

Many types of fragrances can be employed in the present invention, the only limitation being the compatibility with the other components being employed. Suitable fragrances include but are not limited to fruits such as almond, apple, cherry, grape, pear, pineapple, orange, strawberry, raspberry; musk, flower scents such as lavender-like, rose-like, iris-like, carnation-like. Other pleasant scents include herbal and woodland scents derived from pine, spruce and other forest smells. Fragrances may also be derived from various oils, such as essential oils, or from plant materials such as peppermint, spearmint and the like. A list of suitable fragrances is provided in U.S. Pat. No. 4,534,891, the contents of which are incorporated by reference as if set forth in its entirety. Another source of suitable fragrances is found in Perfumes, Cosmetics and Soaps, Second Edition, edited by W. A. Poucher, 1959. Among the fragrances provided in this treatise are acacia, cassie, chypre, cyclamen, fern, gardenia, hawthorn, heliotrope, honeysuckle, hyacinth, jasmine, lilac, lily, magnolia, mimosa, narcissus, freshly-cut hay, orange blossom, orchid, reseda, sweet pea, trefle, tuberose, vanilla, violet, wallflower, and the like.

The term “improving” in the phrase “improving, enhancing or modifying a fragrance formulation” means raising the fragrance formulation to a more desirable character. The term “enhancing” means making the fragrance formulation greater in effectiveness or providing the fragrance formulation with an improved character. The term “modifying” means providing the fragrance formulation with a change in character.

The terms “fragrance formulation”, “fragrance composition”, and “perfume composition” mean the same and refer to a mixture of compounds including, for example, alcohols, aldehydes, ketones, esters, ethers, lactones, nitriles, natural oils, synthetic oils, and mercaptans, which are admixed so that the combined odors of the individual components produce a pleasant or desired fragrance. The fragrance formulation of the present invention is a fragrance formulation comprising a compound of the present invention.

The term “fragrance product” means a consumer product that adds a fragrance or masks a malodor. Fragrance products may include, for example, perfumes, colognes, personal care products such as soaps, shower gels, and hair care products, fabric products, air fresheners, cosmetic preparations, and perfume cleaning agents such as detergents, dishwashing materials, scrubbing compositions, and window cleaners. The fragrance product of the present invention is a consumer product that contains a compound of the present invention.

Olfactory acceptable amount is understood to mean the amount of a compound in a fragrance formulation, wherein the compound will contribute its individual olfactory characteristics. However, the olfactory effect of the fragrance formulation will be the sum of effect of each of the fragrance ingredients. Thus, the compounds of the present invention can be used to improve or enhance the aroma characteristics of the fragrance formulation, or by modifying the olfactory reaction contributed by other ingredients in the formulation. The olfactory acceptable amount may vary depending on many factors including other ingredients, their relative amounts and the olfactory effect that is desired.

The amount of the compounds of the present invention employed in a fragrance formulation varies from about 0.005 to about 50 weight percent, preferably from 0.5 to about 25 weight percent, and more preferably from about 1 to about 10 weight percent. Those with skill in the art will be able to employ the desired amount to provide desired fragrance effect and intensity. In addition to the compounds of the present invention, other materials can also be used in conjunction with the fragrance formulation. Well known materials such as surfactants, emulsifiers, polymers to encapsulate the fragrance can also be employed without departing from the scope of the present invention.

When used in a fragrance formulation, the compounds of the present invention provide unexpected strong floral, muguet, and green characteristics and make the fragrance formulation more desirable and noticeable. The odor qualities found in the compounds of the present invention assist in beautifying and enhancing the finished accord and improve the performance of other materials in the fragrance formulation.

The following are provided as specific embodiments of the present invention. Other modifications of this invention will be readily apparent to those skilled in the art. Such modifications are understood to be within the scope of this invention. As used herein all percentages are weight percent unless otherwise noted, ppm is understood to stand for parts per million, L is understood to be liter, mL is understood to be milliliter, mol is understood to be mole, Kg is understood to be kilogram, g is understood to be gram, and mmHg be millimeters (mm) of mercury (Hg). IFF as used in the examples is understood to mean International Flavors & Fragrances Inc., New York, N.Y., USA.

### EXAMPLE I

Preparation of 4-isobutyl cyclohexanepropanal (Formula II): 4-Isobutyl cyclohexanepropanal was prepared as described in the co-pending, commonly assigned U.S. Ser. No. 12/619,325.

¹HNMR: 0.80-0.96 ppm (m, 8H), 1.02 ppm (t, ˜33% of 2H, J=7.05 Hz), 1.12 ppm (t, ˜67% of 2H, J=7.14 Hz), 1.24-1.35 ppm (m, 4H), 1.43-1.74 ppm (m, 7H), 2.42 ppm (q, 2H, J=7.67 Hz), 9.75-9.77 ppm (m, 1H)

4-Isobutyl cyclohexanepropanal was described as having strong floral, muguet, and green notes.

### EXAMPLE II

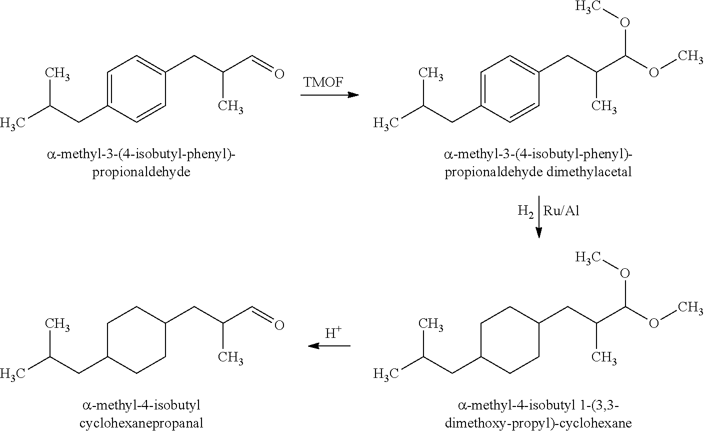

Preparation of α-methyl-4-isobutyl cyclohexanepropanal (Formula III): A 3 L reaction flask was charged with α-methyl-3-(4-isobutyl-phenyl)-propionaldehyde (500 g, 2.5 mol) (commercially available under the trade name Silvial from Givaudan, Inc.), methanol (500 mL), and TMOF (260 g, 2.5 mol). The reaction mass was cooled to 10° C. and hydrochloric acid (37%, 1 g) was added in one portion. The reaction was exothermic and the temperature rose to 23° C. The reaction mass was quenched with sodium methoxide in methanol (25%, 10 g). The crude intermediate product α-methyl-3-(4-isobutyl-phenyl)-propionaldehyde dimethylacetal was isolated by simple distillation (at a boiling point of 92° C. and a pressure of 2 mmHg) and hydrogenated over Ru/Al (10 g) in a 2 L zipper autoclave. The reaction mass was then removed from the autoclave and filtered through celite to remove the catalyst. The crude hydrogenation product (600 g) was placed in a 3 L reaction flask containing acetone (1 L) and aqueous hydrochloric acid (10%, 300 mL). The reaction mass was refluxed for 24 hours and then poured into saturated sodium carbonate solution (1 L). The organic layer was removed. Subsequent fractional distillation afforded α-methyl-4-isobutyl cyclohexanepropanal (394 g) with a boiling point of 92° C. at a pressure of 2 mmHg.

¹HNMR: 9.58 & 9.60 ppm (2d, 1H, J=2.20 Hz & J=2.20 Hz); 2.35-2.47 ppm (m, 1H); 1.43-1.77 ppm (m, 7H); 1.17-1.35 ppm (m, 5H); 1.01-1.15 ppm (m, 5H); 0.85-0.95 ppm (m, 1H); 0.84-0.87 ppm (m, 6H).

α-Methyl-4-isobutyl cyclohexanepropanal was described as having strong floral, muguet, aldehydic, green, and lactonic notes.

### EXAMPLE III

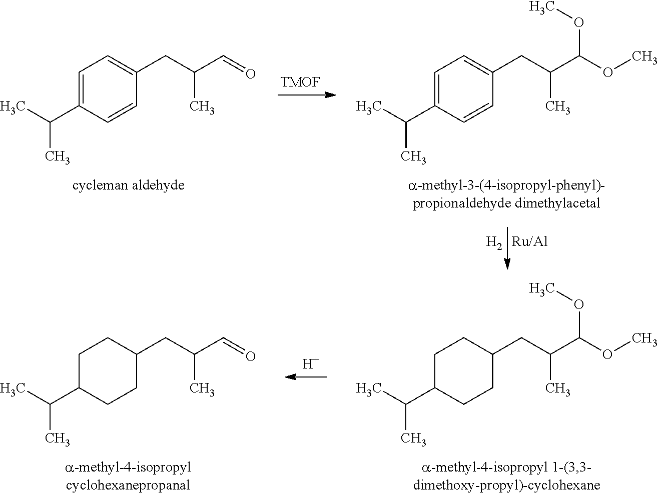

Preparation of α-methyl-4-isopropyl cyclohexanepropanal: α-Methyl-4-isopropyl cyclohexanepropanal, an analog of 4-isobutyl cyclohexanepropanal compounds of the present invention, was also prepared for comparison purposes.

A 3 L reaction flask was charged with cycleman aldehyde (500 g, 2.6 mol) (commercially available from IFF), methanol (500 mL), and TMOF (285 g, 2.7 mol). The reaction mass was cooled to 10° C. and hydrochloric acid (37%, 1 g) was added in one portion. The reaction was exothermic and the temperature rose to 23° C. The reaction mass was quenched with sodium methoxide in methanol (25%, 10 g). The crude intermediate product α-methyl-3-(4-isopropyl-phenyl)-propionaldehyde dimethylacetal was isolated by simple distillation (at a boiling point of 110° C. and a pressure of 1 mmHg) and hydrogenated over Ru/Al (10 g) in a 2 L zipper autoclave. The reaction mass was then removed from the autoclave and filtered through celite to remove the catalyst. The crude hydrogenation product (580 g) was placed in a 3 L reaction flask containing acetone (1 L) and aqueous hydrochloric acid (10%, 300 mL). The reaction mass was refluxed for 24 hours and then poured into saturated sodium carbonate solution (1 L). The organic layer was removed. Subsequent fractional distillation afforded α-methyl-4-isopropyl cyclohexanepropanal (300 g) with a boiling point of 95° C. at a pressure of 5 mmHg.

¹HNMR: 9.58 & 9.61 ppm (2d, 1H, J=2.20 Hz & J=2.20 Hz); 2.35-2.47 ppm (m, 1H); 1.56-1.78 ppm (m, 3H); 1.32-1.52 ppm (m, 6H); 1.16-1.29 ppm (m, 2H); 1.06-1.12 ppm (m, 4H); 0.88-0.99 ppm (m, 1H); 0.83-0.87 ppm (m, 6H).

The compound α-methyl-4-isopropyl cyclohexanepropanal was described as having aldehydic, fresh, and floral notes.

### EXAMPLE IV

α-Methyl-4-isobutyl cyclohexanepropanal (Formula III) and α-methyl-4-isopropyl cyclohexanepropanal were compared at different concentrations for their odor characters using an intensity scale of 0 to 5, where 0=none, 1=minimal, 3=moderate, and 5=intense. The differences in odor profiles and intensity scores were as follows:

| | α-Methyl-4-isobutyl | |
|---|---|---|
| Concentrations | cyclohexanepropanal | α-Methyl-4-isopropyl- |
| (%) | (Formula III) | cyclohexanepropanal |
| 0.01 | Floral, green, lactonic, and more | Barely detectable on blotter, slightly |
| | apparent on blotter; | floral, and slightly fatty |
| | Intensity Score: 1 | Intensity Score: 0.5 |
| 0.1 | Floral, muguet, aldehydic, apparent on | Floral, aldehydic, fatty, lactonic, |
| | blotter, crispy top note, slightly citrusy, | somewhat artificial, and rubbery; |
| | and fresh linen; | Intensity Score: 1 |
| | Intensity Score: 2 | |
| 1.0 | Floral, muguet, clean feel, fresh cut | Floral, muguet like, rubbery-latex, |
| | flower stems, aldehydic, sweet, and | green, aldehydic, somewhat |
| | quite apparent on blotter possessing a | chemical, and green stems; |
| | broader smell with a fresh linen like | Intensity Score: 1.5 |
| | character; | |
| | Intensity Score: 2.5 | |
| 10.0 | Floral, muguet, very apparent on | Floral but with a slightly dirty top |
| | blotter, aldehydic, crispy top note with | note, rubbery green, not very clean; |
| | a very clean olfactive character, and | Intensity Score: 2.5 |
| | representative of the typical muguet | |
| | like floralcy; | |
| | Intensity Score: 4 | |

The above evaluation demonstrated that α-methyl-4-isobutyl cyclohexanepropanal (Formula III) possesses more advantageous and stronger odor characters than α-methyl-4-isopropyl-cyclohexanepropanal.

Formula III displayed a nicer, crisper, and more apparent top note. Formula III was much more performing and provided a fuller and better representation of a floral muguet odor. In contrast, α-methyl-4-isopropyl cyclohexanepropanal was less intense and less clean.

Further, it is known to the skilled in the art that among compounds of different molecular weight, a compound of lower molecular weight would have higher vapor pressure and tend to be more volatile, and, therefore, is expected to possess more intense odor than a compound of higher molecular weight. However, in the current evaluation, Formula III of the present invention has a higher molecular weight than its analog α-methyl-4-isopropyl-cyclohexanepropanal, yet displayed stronger odor intensities at all concentrations tested. The advantageous and superior odor characters of Formula III are thus highly unexpected.

What is claimed is:

1. A compound, α-methyl-4-isobutyl cyclohexanepropanal.

2. A method of improving, enhancing or modifying a fragrance formulation through the addition of an olfactory acceptable amount of α-methyl-4-isobutyl cyclohexanepropanal.

3. The method of claim 2, wherein the fragrance formulation is incorporated into a product selected from the group consisting of a perfume, a cologne, a toilet water, a cosmetic product, a personal care product, a fabric care product, a cleaning product, and an air freshener.

4. The method of claim 3, wherein the cleaning product is selected from the group consisting of a detergent, a dishwashing composition, a scrubbing compound, and a window cleaner.

5. The method of claim 2, wherein the olfactory acceptable amount is from about 0.005 to about 50 weight percent of the fragrance formulation.

6. The method of claim 2, wherein the olfactory acceptable amount is from about 0.5 to about 25 weight percent of the fragrance formulation.

7. The method of claim 2, wherein the olfactory acceptable amount is from about 1 to about 10 weight percent of the fragrance formulation.

8. A fragrance formulation containing an olfactory acceptable amount of α-methyl-4-isobutyl cyclohexanepropanal.

9. The fragrance formulation of claim 8, wherein the olfactory acceptable amount is from about 0.005 to about 50 weight percent of the fragrance formulation.

10. The fragrance formulation of claim 8, wherein the olfactory acceptable amount is from about 0.5 to about 25 weight percent of the fragrance formulation.

11. The fragrance formulation of claim 8, wherein the olfactory acceptable amount is from about 1 to about 10 weight percent of the fragrance formulation.

12. A fragrance product containing an olfactory acceptable amount of the compound of claim 1.
